(19) United States
(12) Patent Application Publication
(10) Pub. No.: US 2005/0054002 A1
(43) Pub. Date: Mar. 10, 2005

(54) Reagent for ante-mortem screening of NCTA

(76) Inventors: Jacques Mayet, Avenheim (FR); Louis Lery, Fontaine Sur Saone (FR)

Correspondence Address: HESLIN ROTHENBERG FARLEY & MESITI PC, 5 COLUMBIA CIRCLE, ALBANY, NY, 12203, US

(21) Appl. No.: 10/651,020

(22) Filed: Aug. 27, 2003

Related U.S. Application Data
(63) Continuation of application No. PCT/FR02/00728, filed on Feb. 28, 2002.

(30) Foreign Application Priority Data
Feb. 27, 2001 (FR) 01.02744

(51) Int. Cl. G01N 33/53

(52) U.S. Cl. 435/7.1

(57) ABSTRACT

Disclosed is a reagent for ante-mortem screening of NCTA obtainable by a method comprising: in a first step, incubating all or part of the 14-3-3 protein or of the anti-14-3-3 protein antibodies in an aqueous aldehyde solution at a final aldehyde dilution of 1 to 2%, in a second step, fixing on a support in the presence of an aqueous aldehyde solution all or part of the 14-3-3 protein or of the anti-14-3-3 protein antibodies contained in the product arising from the first step.

### CROSS-REFERENCE TO RELATED APPLICATIONS

This application is a continuation of PCT International Application No. PCT/FR02/00728 filed Feb. 28, 2002 and published on Sep. 6, 2002 as WO 02/068961 which claims priority of FR 01.02744 filed Feb. 28, 2001. The entire disclosures of the prior applications are incorporated herein by reference.

### FIELD OF THE INVENTION

The invention relates to a reagent for ante-mortem NCTA screening.

It also relates to a method of ante-mortem detection of the presence of NCTA based on said reagent. Moreover, it relates to a method of ante-mortem quantifying NCTA in a biological sample.

### BACKGROUND OF THE INVENTION

Non-conventional transmissible agents (NCTA) or “prions” are responsible for diseases referred to as “transmissible spongiform encephalopathies” (TSE) in humans and animals. Examples of these diseases include Creutzfeld-Jakob disease in humans, scrapie in sheep and goats, and bovine spongiform encephalopathy (BSE) in cattle.

Accurate diagnosis of these diseases, which are fatal since no treatment is available yet, can currently only be performed on biological samples, mainly brain, upon post-mortem examination. Pre-mortem brain biopsy is possible in humans but is not performed anymore because it causes health risks to health-care personnel.

Therefore, there is no available test for human ante-mortem TSE diagnostic without transmission risk.

Thus, there is a need for a reagent for ante-mortem screening of NCTA, without transmission risk.

HSICH et al. in NEJM (1996, 335, vol. 13, pages 924-930 “The 14-3-3 brain protein in cerebrospinal fluid as a marker for transmissible spongiform encephalopathies”) disclosed the systematic presence of an antigenic marker known as the brain “14-3-3 protein” in various biological fluids including cerebrospinal fluid, blood (serum, plasma, leukocyte concentrate), lymph, urine or milk from patients with TSE.

In order to solve the problem mentioned above, the invention proposes a reagent useful for ante-mortem screening of NCTA obtainable by the following method:
in a first step, all or part of the 14-3-3 protein or of the anti-14-3-3 protein antibodies are incubated in an aqueous aldehyde solution at a final aldehyde dilution of 1 to 2%;
in a second step, all or part of the 14-3-3 protein or of the anti-14-3-3 protein antibodies contained in the product arising from the first step are fixed on a support in the presence of an aqueous aldehyde solution.

In other words, the invention relates to a reagent mainly consisting in supports on which all or part of the anti-14-3-3 protein antibodies or all or part of the 14-3-3 proteins, commercially available and artificially synthesised, are immobilized. Aldehyde has a distinct role in each step, respectively:
in the first step, it increases the efficiency of the titration and improves the test sensitivity without altering the antigenic properties of the antibodies or proteins,
in the second step, it helps the coupling between the antibodies or proteins and the support.

In order not to alter the antigenic properties of the 14-3-3 proteins or of the anti-14-3-3 protein antibodies, the incubation time between these elements and the aldehyde solution in the first step is preferably inferior to 1 hour, preferably superior to 15 minutes.

The aldehyde used in practice is glutaraldehyde.

In practice, the supports on which the anti-14-3-3 protein antibodies or the 14-3-3 proteins are fixed are red blood cells, specially from avian source, or artificial membranes.

According to another characteristic of this method, the aldehyde concentration in the aqueous solution used in the second step is comprised between 0.01 and 0.1 M.

In a preferred embodiment, a step of washing the product arising from the first step is introduced between the first and second steps.

The invention also concerns a method of ante-mortem detecting the presence of NCTA in a biological sample.

This method consists in the incubation of the biological sample with the reagent as described above, and in the detection by agglutination and/or agglutination inhibition of the presence or absence of all or part of the anti-14-3-3 protein antibodies or all or part of the 14-3-3 proteins in said biological sample.

As already mentioned, the biological sample is preferably selected among the group of biological fluids including: cerebrospinal fluid, blood (serum, plasma, leukocyte concentrate), lymph, urine or milk.

In one embodiment, wherein the supports or artificial membranes are impregnated with markers, i.e. with the 14-3-3 protein, the detection of an agglutination by the anti-14-3-3 protein antibodies, after incubation with the biological sample, reveals the absence of antigens in the blood, and then the absence of infection. In contrast, no agglutination reveals the presence of antigens in the biological sample, and then the presence of an infection.

In another embodiment wherein the supports or artificial membranes are impregnated with the anti-14-3-3 protein antibodies, a direct agglutination of said supports by the 14-3-3 protein reveals the presence of antigens in the biological sample, and therefore an infection.

The absence of agglutination can be linked to an insufficient amount of marker in the sample. In this case, an excess of the 14-3-3 protein is added in the biological sample. The absence of agglutination then reveals the presence of antigens. In contrast, the detection of an agglutination reveals the absence of antigens.

The invention also relates to a method of ante-mortem quantifying NCTA in a biological sample. According to this method, the biological sample of interest is incubated at various concentrations with the reagent as described above, the dilution for which agglutination and/or agglutination inhibition occurs is determined and then the concentration of the 14-3-3 proteins or of the anti-14-3-3 protein antibodies is deduced by comparison with a standard curve.

The following example is provided to illustrate the invention and its benefits.

### PREPARATION OF THE AVIAN RED BLOOD CELLS IMPREGNATED WITH THE 14-3-3 PROTEIN

The 14-3-3 protein is prepared in a PBS buffer solution containing glutaraldehyde at a final dilution of 1.5%. The proteins and the glutaraldehyde solution are incubated during 20 minutes.

In parallel, turkey red blood cells are suspended in a PBS buffer solution at pH 7.4, in a ratio of 5 ml of red blood cells to 100 ml PBS.

To this suspension is then added an aqueous solution of glutaraldehyde of concentration 0.05 M and the protein solution arising from the first step.

The incubation between the red blood cells, glutaraldehyde and the proteins lasts 10 minutes. The red blood cells impregnated with the proteins and collected by centrifugation are then washed and resuspended in a PBS buffer solution. A lysine solution is then added to the suspension for 15 to 30 minutes.

The red blood cells impregnated and treated are then collected by centrifugation, washed and resuspended in a PBS solution containing 0.7/10 000 (weight/volume) sodium azide.

This reagent was used in sheep, in which scrapie represents one form of spongiform encephalopathies. It resulted in a positive reaction in 4 cases over 5, when applied to the cerebrospinal fluid sampled just after the death. The parallel test was negative when performed on 2 healthy animals.

The invention and its benefits clearly appear from the description.

The possibility of an ante-mortem diagnostic for prion diseases in humans and animals by a simple and reliable test without transmission risks is remarkable.

1. A reagent for ante-mortem screening of NCTA obtainable by a method comprising:
in a first step, all or part of the 14-3-3 protein or of the anti-14-3-3 protein antibodies are incubated in an aqueous aldehyde solution at a final aldehyde dilution of 1 to 2%;
in a second step, all or part of the 14-3-3 protein or of the anti-14-3-3 protein antibodies contained in the product arising from the first step are fixed on a support in the presence of an aqueous aldehyde solution.

2. The reagent of claim 1 wherein, in the first step, the incubation time between the 14-3-3 protein or the anti-14-3-3 protein antibodies and the aqueous aldehyde solution is less than 60 minutes, preferably more than 15 minutes.

3. The reagent of claim 1 wherein the aldehyde concentration of the aqueous solution used in the second step is between about 0.01 and 0.1 M.

4. The reagent of claim 1 wherein a step of washing the product arising from the first step is introduced between the first and second steps.

5. The reagent of claim 1 wherein the support consists of avian red blood cells or artificial membranes.

6. The reagent of claim 1 wherein the aldehyde is glutaraldehyde.

7. A method for ante-mortem screening of NCTA comprising: a) incubating a reagent according to claim 1 with a biological sample and b) detecting by agglutination and/or agglutination inhibition of the presence or absence of anti-14-3-3 protein antibodies or 14-3-3 proteins in said biological sample.

8. A method according to claim 7 wherein the biological sample consists in a biological fluid selected from cerebrospinal fluid, blood (serum, plasma, leukocyte concentrate), lymph, urine or milk.

9. A method of ante-mortem quantifying NCTA wherein a biological sample of interest is incubated at various concentrations with the reagent according to claims 1 to 6, the dilution for which agglutination and/or agglutination inhibition occurs is determined and then the concentration of the 14-3-3 proteins or of the anti-14-3-3 protein antibodies is deduced by comparison with a standard curve.
